(19) United States
(12) Patent Application Publication
(10) Pub. No.: US 2014/0018820 A1
(43) Pub. Date: Jan. 16, 2014

(54) PALPATION APPARATUS AND METHOD USING ROBOT

(71) Applicant: SAMSUNG ELECTRONICS CO., LTD., Suwon-si (KR)

(72) Inventors: Soo Chul LIM, Seoul (KR); Joon Ah PARK, Hwasung-si (KR); Hyung Kew LEE, Gunpo-si (KR); Bho Ram LEE, Sungnam-si (KR); Seung Ju HAN, Seoul (KR); Hyun Jeong LEE, Yongin-si (KR)

(73) Assignee: Samsung Electronics Co., Ltd., Suwon-si (KR)

(21) Appl. No.: 13/746,736

(22) Filed: Jan. 22, 2013

(30) Foreign Application Priority Data
Jul. 11, 2012 (KR) 10-2012-0075664

(51) Int. Cl. A61B 19/00

(52) U.S. Cl. CPC A61B 19/20
USPC 606/130

(57) ABSTRACT

A palpation apparatus and method using a robot may include a motion controller to control a motion of the robot based on an input of a user, and a force measuring unit to measure a magnitude of a reaction force of an object in contact with the robot based on the motion, through a sensor attached to the robot.

### CROSS-REFERENCE TO RELATED APPLICATIONS

This application claims the priority benefit of Korean Patent Application No. 10-2012-0075664, filed on Jul. 11, 2012, in the Korean Intellectual Property Office, the disclosure of which is incorporated herein by reference.

BACKGROUND

1. Field

The following description relates to a palpation apparatus and method using a robot, and more particularly, to an apparatus and method that may provide palpation information to a user when a surgical robot is controlled remotely by the user.

2. Description of the Related Art

While performing surgery on a patient, a medical doctor may discover a disease or a wound that was not perceived before the surgery is performed, by palpating an organ of the patient with a hand.

However, palpation using a surgical robot may be impossible because the doctor may perform the surgery using an image captured by a camera, without contact with the patient. Accordingly, the doctor may face difficulties in discovering a wound that is narrowly perceived through an image, or a disease that is not perceived before the surgery, using a conventional surgical robot.

Thus, there is a demand for a method of performing palpation using a surgical robot.

### SUMMARY

The foregoing and/or other aspects are achieved by providing a palpation apparatus using a robot, the palpation apparatus including a motion controller to control a motion of the robot based on an input of a user, and a force measuring unit to measure a magnitude of a reaction force of an object in contact with the robot based on the motion, through a sensor attached to the robot.

The palpation apparatus may further include a feedback determining unit to determine a magnitude and a direction of a feedback to be provided to the user, based on the magnitude and a direction of the reaction force of the object.

The palpation apparatus may further include a property value determining unit to determine a property value of the object, based on the magnitude and the direction of the reaction force of the object, a state determining unit to determine a state of the object, based on reference information indicating state information of the object based on property values of the object, and the determined property value of the object, and a state displaying unit to display, to the user, the state of the object, or a name of a disease related to the state of the object.

The force measuring unit may measure a magnitude of a force fed back to a gripper of the robot by an object being gripped by the gripper, through a sensor attached to an internal side of the gripper gripping the object, and may measure a magnitude of a force fed back to an external side of a gripper of the robot by an object in contact with the external side of the gripper, through a sensor attached to the external side of the gripper.

The force measuring unit may measure a magnitude of a force fed back to a probe of the robot by the object, or a shape of a surface of the object, through a sensor attached to the probe.

The foregoing and/or other aspects are achieved by providing a palpation method using a robot, the palpation method including controlling a motion of a robot based on an input of a user, and measuring a magnitude of a reaction force of an object in contact with the robot based on the motion, through a sensor attached to the robot.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects will become apparent and more readily appreciated from the following description of embodiments, taken in conjunction with the accompanying drawings of which:

FIG. 1 illustrates a surgical system according to example embodiments;

FIG. 2 illustrates a structure of a palpation apparatus of FIG. 1;

FIG. 3 illustrates a process of measuring a magnitude of a force fed back by an object being gripped by a gripper, through a sensor attached to an external side of the gripper according to example embodiments;

FIG. 4 illustrates a process of measuring a magnitude of a force fed back by an object in contact with a gripper, through a sensor attached to an external side of the gripper according to example embodiments;

FIG. 5 illustrates a process of measuring a magnitude of a force fed back by an object being gripped by a gripper, through a sensor attached to an internal side of the gripper according to example embodiments;

FIG. 6 illustrates a process of measuring a magnitude of a force fed back by an object in contact with a gripper, through a sensor attached to a tip of the gripper according to example embodiments;

FIG. 7 illustrates a process of measuring a magnitude of a force fed back by an object in contact with a probe, through a sensor attached to the probe according to example embodiments;

FIG. 8 illustrates a process of measuring a magnitude of a force fed back by an object in contact with a flexible arm, through a sensor attached to the flexible arm according to example embodiments;

FIG. 9 illustrates a robot including a gripper and a probe independent from one another, according to example embodiments; and

FIG. 10 illustrates a palpation method using a robot according to example embodiments.

### DETAILED DESCRIPTION

Reference will now be made in detail to embodiments, examples of which are illustrated in the accompanying drawings, wherein like reference numerals refer to the like elements throughout. Embodiments are described below to explain the present disclosure by referring to the figures.

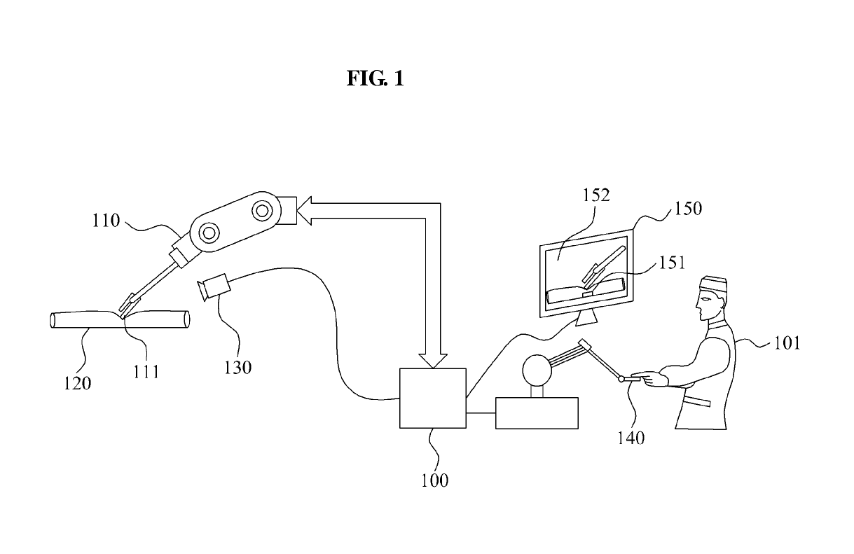

FIG. 1 illustrates a surgical system according to example embodiments.

As illustrated in FIG. 1, a user 101 may input a control command into controllers 140 with both hands while viewing, on a display 150, an image 152 captured by a camera 130. A palpation apparatus 100 may control a robot 110 to be in contact with an object 120 or to control the object 120, based on the control command input into the controllers 140. In this instance, the object 120 may correspond to an organ, an area of skin, or a surgical site of a patient, for example.

The palpation apparatus 100 may palpate the object 120 through a sensor 111 attached to the robot 110, and may display a result of the palpation in the image 152.

In particular, the palpation apparatus 100 may identify a property value of the object 120, based on a magnitude of a reaction force of the object 120 in contact with the robot 110. The palpation apparatus 100 may identify a wound in the patient based on the property value of the object 120, or the magnitude of the reaction force of the object 120, thereby generating a result of palpation identical to a result which may be obtained when the user 101 palpates the object 120 directly.

The palpation apparatus 100 may display the identified wound of the object 120 in the image 152, thereby enabling the user 101 to perceive a location and a state of the wound, in a visual manner.

The palpation apparatus 100 may display the property value of the object 120, or the magnitude of the reaction force of the object 120, using a value 151, thereby enabling the user 101 to perceive the property value of the object 120, or the magnitude of the reaction force of the object 120, in a visual manner.

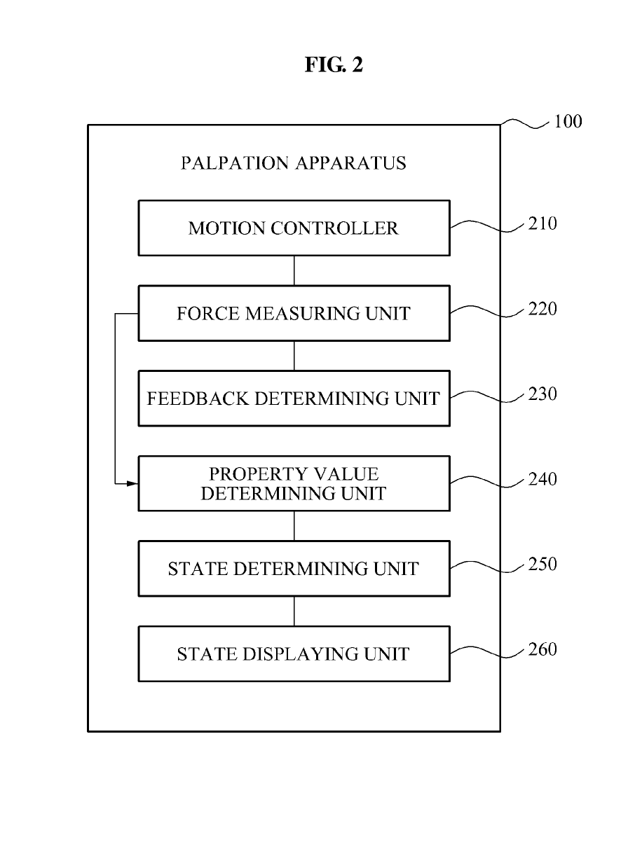

FIG. 2 illustrates a structure of the palpation apparatus 100 of FIG. 1.

Referring to FIG. 2, the palpation apparatus 100 may include a motion controller 210, a force measuring unit 220, a feedback determining unit 230, a property value determining unit 240, a state determining unit 250, and a state displaying unit 260.

The motion controller 210 may control a motion of the robot 110 to be in contact with the object 120, based on an input of the user 101. In particular, the motion controller 210 may control the motion of the robot 110, based on a control command input into the controllers 140 by the user 101.

In this instance, the robot 110 may include a gripper to grip and control the object 120, for example an organ, or a surgical instrument, and an arm to manipulate a position of the gripper. In addition, the robot 110 may include a probe to be in contact with the object 120, and an arm to manipulate a position of the probe. In this instance, the aforementioned arms may correspond to flexible arms which may include a plurality of joints, and may bend in various directions to form various shapes.

The force measuring unit 220 may measure a magnitude of a reaction force of the object in contact with the robot 110, through the sensor 111 attached to the robot 110. In this instance, the sensor 111 may correspond to a three-axis sensor that may measure a magnitude of a reaction force of the object 120, with respect to each of an X-axial direction, a Y-axial direction, and a Z-axial direction.

The sensor 111 may be attached to an internal side of the gripper, an external side of the gripper, a tip of the gripper, the arm, or the probe of the robot 110. In this instance, the internal side of the gripper may refer to a side to be in contact with the object 120 when the gripper grips the object 120, and the external side of the gripper may refer to remaining sides, excluding the internal side from the gripper.

In this instance, the force measuring unit 220 may measure, through a sensor attached to the external side of the gripper of the robot 110, a magnitude of a force fed back to the external side of the gripper by the object 120 in contact with the external side of the gripper, or a magnitude of a force fed back to the gripper by the object 120 being gripped by the gripper.

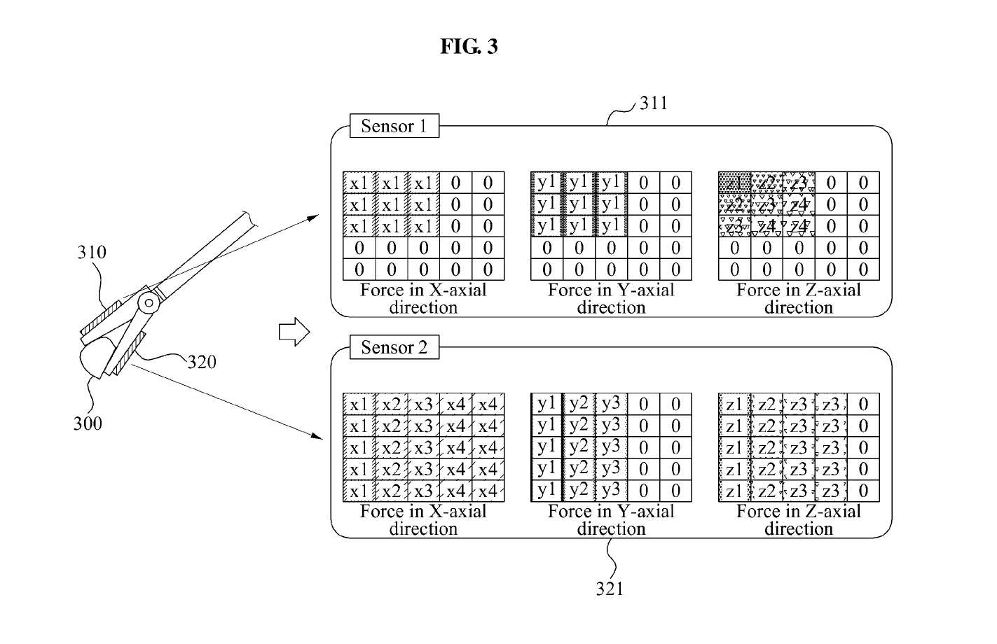

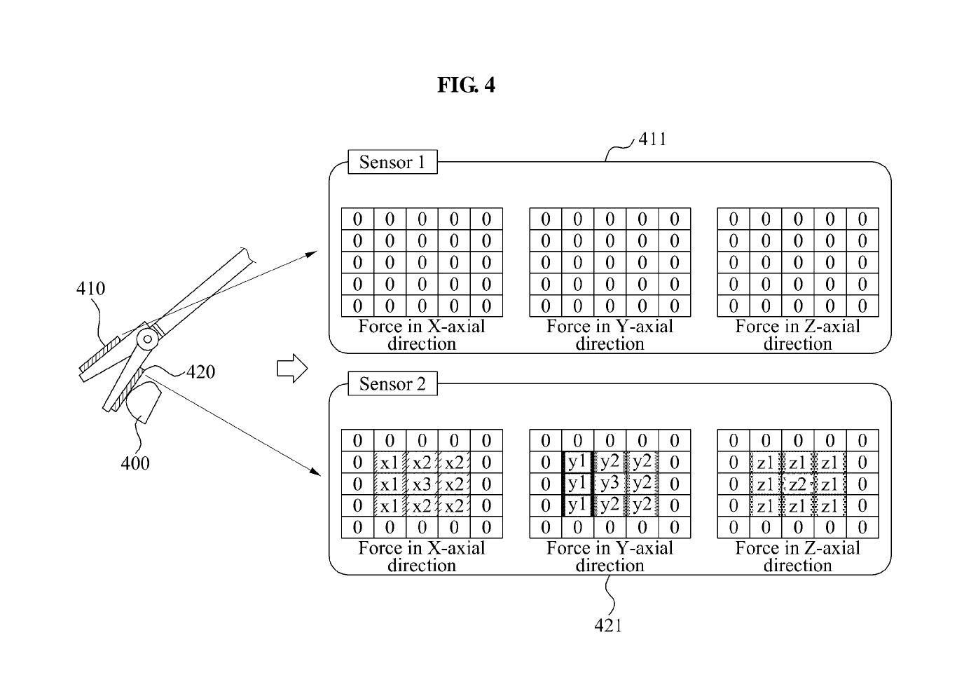

Example embodiments of the force measuring unit 220 measuring the magnitude of the fed-back force, through the sensor attached to the external side of the gripper will be described in detail with reference to FIGS. 3 and 4.

In addition, the force measuring unit 220 may measure a magnitude of a force fed back to the gripper by the object 120 being gripped by the gripper, through a sensor attached to the internal side of the gripper of the robot 110.

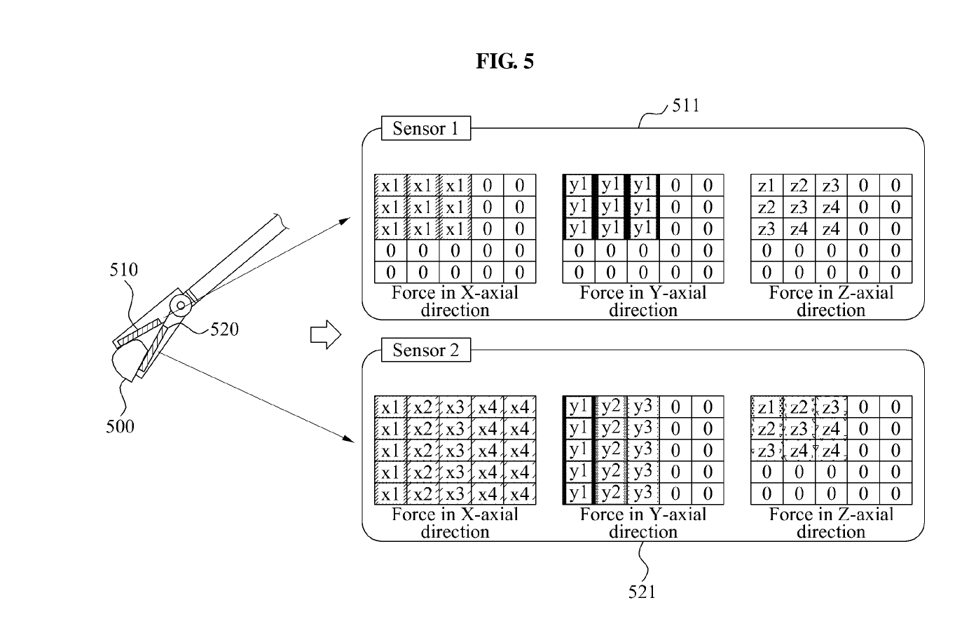

Example embodiments of the force measuring unit 220 measuring the magnitude of the fed-back force, through the sensor attached to the internal side of the gripper will be described in detail with reference to FIG. 5.

Further, the force measuring unit 220 may measure a magnitude of a force fed back to the probe by the object 120, or a shape of a surface of the object 120, through a sensor attached to the probe of the robot 110.

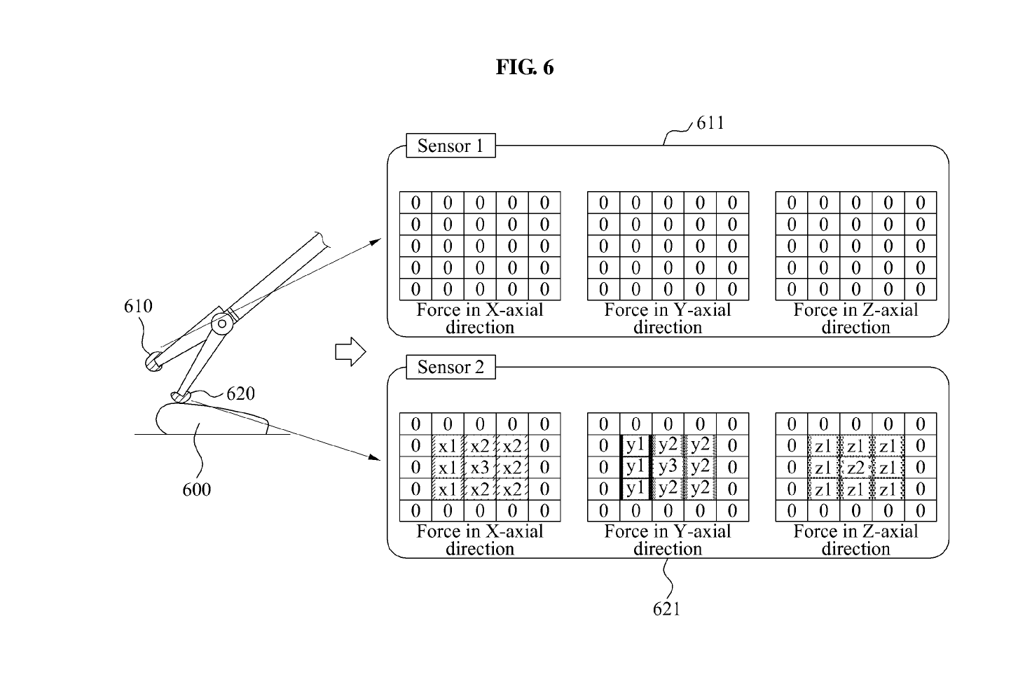

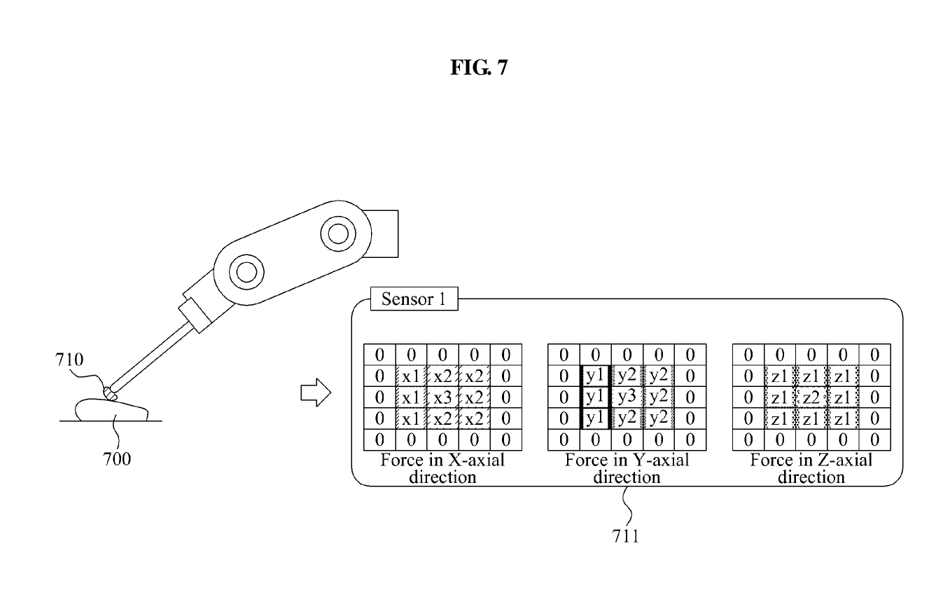

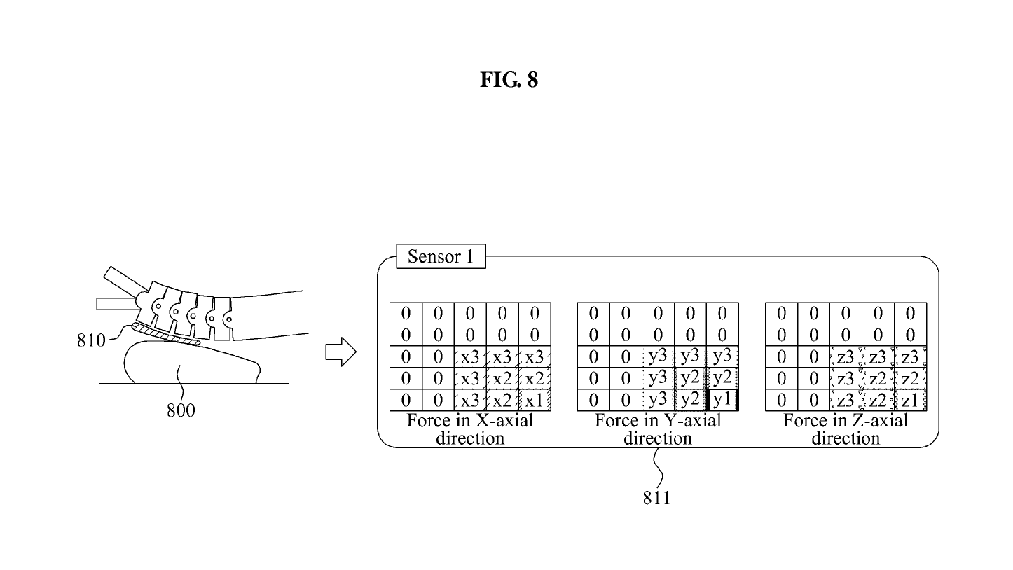

Example embodiments of the force measuring unit 220 measuring the magnitude of the fed-back force, through the sensor attached to the tip of the gripper or the probe will be described in detail with reference to FIGS. 6 and 7. In addition, example embodiments of the force measuring unit 220 measuring the magnitude of the fed-back force, through a sensor attached to the arm, will be described in detail with reference to FIG. 8.

The feedback determining unit 230 may determine a magnitude and a direction of a feedback to be provided to the user 101, based on a magnitude and a direction of the reaction force of the object 120. In this instance, the feedback determining unit 230 may control the controllers 140 based on the determined magnitude and the determined direction of the feedback, thereby providing the user 101 with a force at a magnitude identical to the magnitude of the reaction force of the object 120, and in a direction identical to the direction of the reaction force of the object 120.

That is, the feedback determining unit 230 may provide, through the controllers 140, a sensation identical to a sensation perceived by the user 101 gripping the object 120 directly with his or her hands.

In this instance, the controllers 140 may provide feedback of a force or a pressure back to fingers of the user 101, using an array actuator provided in a structure in which a kinesthetic feedback may be provided and the fingers may be transformed. In addition, the controllers 140 may provide the user 101 with an oscillating array feedback, using an array actuator that may move rapidly.

The property value determining unit 240 may determine a property value of the object 120, based on the magnitude and the direction of the reaction force of the object 120.

When the robot 110 applies a force to the object 120 at an identical magnitude, the object 120 may provide feedback of a force back to the robot 110 at different magnitudes and in different directions, depending on property values such as elasticity, for example. Accordingly, the property value determining unit 240 may determine the property value of the object 120, by comparing the force applied to the object 120 by the robot 110 to the reaction force of the object 120.

The state determining unit 250 may determine a state of the object 120, based on reference information, and the property value of the object 120 determined by the property value determining unit 240. In this instance, the reference information may refer to state information indicating a state of the object 120 when the property value of the object 120 is within a predetermined range.

For example, an inflamed site of the object 120 may have a relatively low value of elasticity due to inflammation of the site. In this instance, the reference information of the object 120 may include information indicating that the object 120 is normal when the property value of the object 120 corresponds to a reference value, and information indicating that the object 120 is inflamed when the property value of the object 120 is lower than the reference value. Accordingly, when the property value of the object 120 is lower than the reference value, the state determining unit 250 may determine that a portion of the object 120 which is in contact with the robot 110 is inflamed.

The state displaying unit 260 may display the magnitude and the direction of the reaction force of the object 120, in a visual manner. In this instance, the state displaying unit 260 may display a magnitude and a direction of a force identical to the feedback to be provided to the user 101 by the feedback determining unit 230, in a portion of the image 152 displaying the object 120, using the value 151 or a graph.

In addition, the state displaying unit 260 may display, to the user, the state of the object 120 determined by the state determining unit 250. For example, when the state determining unit 250 determines that the site of the object 120 is inflamed, the state displaying unit 260 may display the wound in the object 120 displayed in the image 152. In this instance, the state displaying unit 260 may display a message indicating the object 120 is inflamed, in the image 152.

FIG. 3 illustrates a process of measuring a magnitude of a force fed back by an object being gripped by a gripper, through a sensor attached to an external side of the gripper according to example embodiments.

Referring to FIG. 3, when a gripper grips an object 300, the force measuring unit 220 may measure a magnitude of a force fed back to the gripper by the object 300, through a first sensor 310 and a second sensor 320 that are attached to external sides of the gripper. Also, when the gripper pulls the object 300, the force measuring unit 220 may measure a magnitude of a force fed back to the gripper by the object 300, through the first sensor 310 and the second sensor 320 attached to the external sides of the gripper. In this instance, the property value determining unit 240 may determine a property value of the object 300, based on the magnitude of the force measured by the force measuring unit 220. The state determining unit 250 may determine a presence of a wound in a patient, based on the property value determined by the property value determining unit 240.

In this instance, the first sensor 310 and the second sensor 320 may measure the magnitude of the force fed back to the gripper by the object 300, with respect to each of an X-axial direction, a Y-axial direction, and a Z-axial direction. Because the second sensor 320 is positioned under the object 300, a magnitude of a force 321 to be measured by the second sensor 320 may include the magnitude of the force fed back to the gripper by the object 300, and a magnitude of a force applied to the gripper corresponding to a weight of the object 300. Accordingly, due to the weight of the object 300, the magnitude of the force 321 measured by the second sensor 320 may be greater than a magnitude of a force 311 measured by the first sensor 310.

FIG. 4 illustrates a process of measuring a magnitude of a force fed back by an object in contact with a gripper, through a sensor attached to an external side of the gripper according to example embodiments.

Referring to FIG. 4, when the external side of the gripper is in contact with an object 400, based on a control of the motion controller 210 of FIG. 2, the force measuring unit 220 may measure a magnitude of a force fed back to the gripper by the object 400, through a second sensor 420 attached to the external side of the gripper. In this instance, the property value determining unit 240 may determine a property value of the object 400, based on the magnitude of the force measured by the force measuring unit 220. The state determining unit 250 may determine a presence of a wound in a patient, based on the property value determined by the property value determining unit 240.

In this instance, a first sensor 410 and the second sensor 420 attached to external sides of the gripper may measure the magnitude of the force fed back to the gripper by the object 400, with respect to each of an X-axial direction, a Y-axial direction, and a Z-axial direction. However, because the first sensor 410 is not in contact with the object 400, a magnitude of a force 411 measured by the first sensor 410 may correspond to “0” in each respective direction, as shown in FIG. 4. The second sensor 420 may measure a magnitude of a force 421 fed back to the gripper by the object 400, with respect to each of an X-axial direction, a Y-axial direction, and a Z-axial direction based on a location at which the second sensor 420 is in contact with the object 400.

FIG. 5 illustrates a process of measuring a magnitude of a force fed back by an object being gripped by a gripper, through a sensor attached to an internal side of the gripper according to example embodiments.

Referring to FIG. 5, when the gripper grips an object 500, the force measuring unit 220 of FIG. 2 may measure a magnitude of a force fed back to the gripper by the object 500, through a first sensor 510 and a second sensor 520 that are attached to internal sides of the gripper. In this instance, the property value determining unit 240 may determine a property value of the object 500, based on the magnitude of the force measured by the force measuring unit 220. The state determining unit 250 may determine a presence of a wound in a patient, based on the property value determined by the property value determining unit 240.

In this instance, the first sensor 510 and the second sensor 520 may measure the magnitude of the force fed back to the gripper by the object 500 in response to a grip strength of the gripper, with respect to each of an X-axial direction, a Y-axial direction, and a Z-axial direction. Because the second sensor 520 is positioned under the object 500, a magnitude of a force 521 to be measured by the second sensor 520 may include the magnitude of the force fed back to the gripper by the object 500, and a magnitude of a force applied to the gripper corresponding to a weight of the object 500. Accordingly, due to the weight of the object 500, the magnitude of the force 521 measured by the second sensor 520 may be greater than a magnitude of a force 511 measured by the first sensor 510.

FIG. 6 illustrates a process of measuring a magnitude of a force fed back by an object in contact with a gripper, through a sensor attached to a tip of the gripper according to example embodiments.

Referring to FIG. 6, when the tip of the gripper is in contact with an object 600 based on a control of the motion controller 210 of FIG. 2, the force measuring unit 220 may measure a magnitude of a force fed back to the gripper by the object 600, through a second sensor 620 attached to the tip of the gripper. In this instance, the property value determining unit 240 may determine a property value of the object 600, based on the magnitude of the force measured by the force measuring unit 220. The state determining unit 250 may determine a presence of a wound in a patient, based on the property value determined by the property value determining unit 240.

In addition, a first sensor 610 and the second sensor 620 may measure the magnitude of the force fed back to the gripper by the object 600, with respect to each of an X-axial direction, a Y-axial direction, and a Z-axial direction. However, because the first sensor 610 is not in contact with the object 600, a magnitude of a force 611 measured by the first sensor 610 may correspond to “0” in each respective direction, as shown in FIG. 6. The second sensor 620 may measure a magnitude of a force 621 fed back to the gripper by the object 600, with respect to each of an X-axial direction, a Y-axial direction, and a Z-axial direction based on a location at which the second sensor 620 is in contact with the object 600.

FIG. 7 illustrates a process of measuring a magnitude of a force fed back by an object in contact with a probe, through a sensor attached to the probe according to example embodiments.

A robot may include the probe, rather than a gripper, to measure a magnitude of a force fed back by an object, as shown in FIG. 7. In this instance, a sensor 710 may measure the magnitude of the force fed back to the probe by an object 700, with respect to each of an X-axial direction, a Y-axial direction, and a Z-axial direction.

When a tip of the probe is in contact with the object 700 based on a control of the motion controller 210 of FIG. 2, the force measuring unit 220 may measure a magnitude of a force 711 fed back to the probe by the object 700, through the sensor 710 attached to the tip of the probe. In this instance, the property value determining unit 240 may determine a property value of the object 700, based on the magnitude of the force measured by the force measuring unit 220. The state determining unit 250 may determine a presence of a wound in a patient, based on the property value determined by the property value determining unit 240.

In addition, the force measuring unit 220 may measure a shape of a surface of the object 700, through the sensor 710 attached to the tip of the probe. In particular, when the probe is moved at an identical height along the surface of the object 700, a relatively great magnitude of pressure may be applied to a part protruding from the surface of the object 700 by the probe. Accordingly, such a protruding part may provide a feedback having greater magnitude of a force back to the probe than magnitudes of forces fed back by other parts. In addition, a relatively small magnitude of pressure may be applied to a part with a void on the surface of the object 700 by the probe. Accordingly, such a void may provide a feedback having smaller magnitude of a force back to the probe than magnitudes of forces fed back by other parts.

Accordingly, the force measuring unit 220 may store a change in the magnitude of the force measured by the sensor 710 when the probe moves, and may estimate a curvature of the surface of the object 700 based on the change in the magnitude of the force, thereby measuring the shape of the surface of the object 700.

FIG. 8 illustrates a process of measuring a magnitude of a force fed back by an object in contact with a flexible arm, through a sensor attached to the flexible arm according to example embodiments.

A robot may control a position of a probe or a gripper, through the flexible arm, as shown in FIG. 8.

Referring to FIG. 8, the flexible arm may be formed by connecting a plurality of joints consecutively, and may change a shape or an area of a surface to be in contact with an object 800. For example, in FIG. 8, because the flexible arm bends upwards, an area of the surface in contact with the object 800 may broaden, and a shape of the surface in contact with the object 800 may be convex. Conversely, when the flexible arm bends downwards, the area of the surface in contact with the object 800 may narrow, and the shape of the surface in contact with the object 800 may be concave.

The robot may include a sensor 810 attached to one side or both sides of the flexible arm, as shown in FIG. 8. In this instance, the force measuring unit 220 of FIG. 2 may measure a magnitude of a force 811 fed back to the flexible arm in contact with the object 800, through the sensor 810. In addition, the property value determining unit 240 may determine a property value of the object 800, based on the magnitude of the force measured by the force measuring unit 220. The state determining unit 250 may determine a presence of a wound in a patient, based on the property value determined by the property value determining unit 240.

The sensor 810 may measure the magnitude of the force fed back to the flexible arm by the object 800, with respect to each of an X-axial direction, a Y-axial direction, and a Z-axial direction.

When the gripper or the probe is not in contact with the object 800, or fails to be in contact with the object 800, the robot according to example embodiments may control the flexible arm, which controls the position of the gripper or the probe, to be in contact with the object 800, and may palpate the object 800 through the sensor 810 attached to the flexible arm.

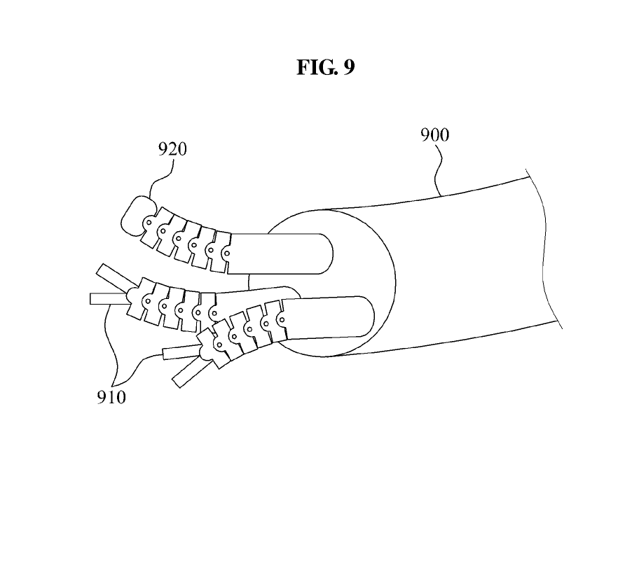

FIG. 9 illustrates a robot including a gripper and a probe independently according to example embodiments.

FIG. 9 illustrates an example embodiment of a single flexible arm 900 including a plurality of surgical instruments.

In this instance, the flexible arm 900 may include a gripper 910 that grips an object, and a probe 920 that palpates the object.

In particular, the flexible arm 900 may control the gripper 910 to grip or cut the object, based on a control of the motion controller 210 of FIG. 2. In this instance, the flexible arm 900 may control the probe 920 to be in contact with the object being gripped by the gripper 910.

In this instance, the force measuring unit 220 may measure a magnitude of a force fed back to the probe 920 by the object, through a sensor attached to the probe 920, and may estimate a minute shape of a surface of the object based on the measured magnitude of the force. In addition, the property value determining unit 240 may determine a property value of the object, based on the magnitude of the force measured by the force measuring unit 220. The state determining unit 250 may determine a presence of a wound or a name of a disease, based on the property value determined by the property value determining unit 240.

The flexible arm 900 may include the gripper 910 that controls the object, and the probe 920 that measures the magnitude of the force fed back by the object, independently, thereby measuring a property value of another part different from a part in contact with the gripper 910 while a user controls the object with the gripper 910.

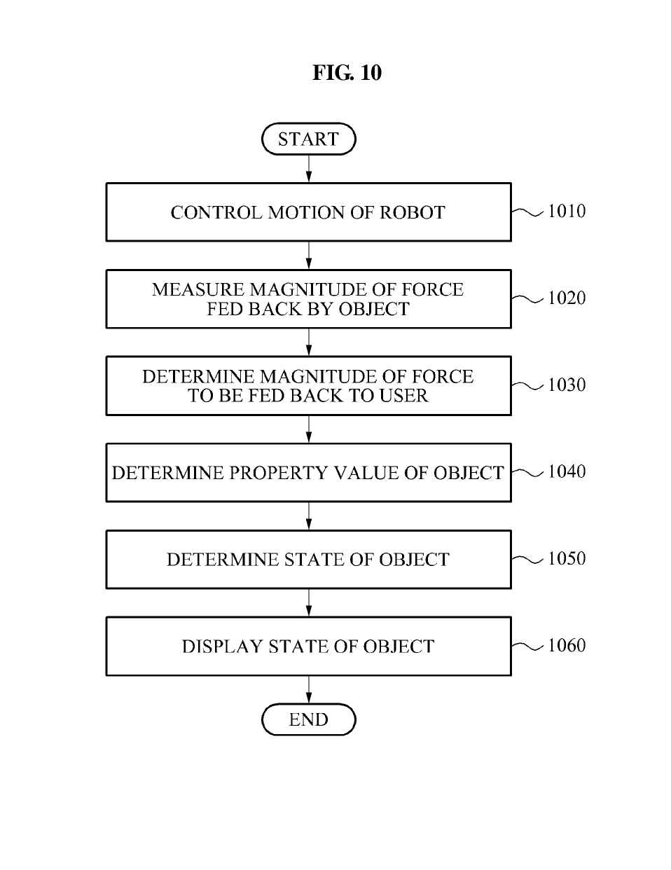

FIG. 10 illustrates a palpation method using a robot according to example embodiments. The palpation method of FIG. 10 will be described with reference to FIGS. 1 and 2.

In operation 1010, the motion controller 210 may control a motion of the robot 110 to be in contact with the object 120, based on an input of a user.

In operation 1020, the force measuring unit 220 may measure a magnitude of a reaction force of the object 120, which is in contact with the robot 110 in operation 1010, through the sensor 111, for example, a three-axis sensor, attached to the robot 110.

In operation 1030, the feedback determining unit 230 may determine a magnitude and a direction of a feedback to be provided to the user, based on the magnitude and a direction of the force measured in operation 1020. In this instance, feedback determining unit 230 may control the controllers 140 based on the determined magnitude and the determined direction of the feedback, thereby providing the user with a force at a magnitude identical to the magnitude of the reaction force of the object 120, and in a direction identical to the direction in which the force is fed back to the robot 110 by the object 120.

In operation 1040, the property value determining unit 240 may determine a property value of the object 120, based on the magnitude and the direction of the force measured in operation 1020.

In operation 1050, the state determining unit 250 may determine a state of the object 120, based on reference information, and the property value of the object 120 determined in operation 1040. In this instance, the reference information may refer to state information indicating a state of the object 120 when the property value of the object 120 is within a predetermined range.

In operation 1060, the state displaying unit 260 may display the state of the object 120 determined in operation 1050. In addition, the state displaying unit 260 may display the magnitude and the direction of the force measured in operation 1020, in a visual manner.

The method according to the above-described embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The described hardware devices may be configured to act as one or more software modules in order to perform the operations of the above-described embodiments, or vice versa.

Although embodiments have been shown and described, it would be appreciated by those skilled in the art that changes may be made in these embodiments without departing from the principles and spirit of the disclosure, the scope of which is defined by the claims and their equivalents.

What is claimed is:

1. A palpation apparatus using a robot, the apparatus comprising:
a motion controller to control a motion of the robot based on an input of a user; and
a force measuring unit to measure a magnitude of a reaction force of an object in contact with the robot based on the motion, through a sensor attached to the robot.

2. The apparatus of claim 1, further comprising:
a feedback determining unit to determine a magnitude and a direction of a feedback to be provided to the user, based on the magnitude and a direction of the reaction force of the object.

3. The apparatus of claim 1, further comprising:
a property value determining unit to determine a property value of the object, based on the magnitude and a direction of the reaction force of the object;
a state determining unit to determine a state of the object, based on reference information indicating state information of the object based on property values of the object, and the determined property value of the object; and
a state displaying unit to display, to the user, the state of the object, or a name of a disease related to the state of the object.

4. The apparatus of claim 3, wherein the state displaying unit displays the magnitude and the direction of the reaction force of the object, in a visual manner.

5. The apparatus of claim 1, wherein the force measuring unit measures a magnitude of a force fed back to a gripper of the robot by an object being gripped by the gripper, through a sensor attached to an internal side of the gripper gripping the object.

6. The apparatus of claim 1, wherein the force measuring unit measures a magnitude of a force fed back to an external side of a gripper of the robot by an object in contact with the external side of the gripper, through a sensor attached to the external side of the gripper.

7. The apparatus of claim 1, wherein the force measuring unit measures a magnitude of a force fed back to a probe of the robot by the object, or a shape of a surface of the object, through a sensor attached to the probe.

8. The apparatus of claim 1, wherein the sensor measures the magnitude of the reaction force of the object, with respect to each of an X-axial direction, a Y-axial direction, and a Z-axial direction.

9. A palpation method using a robot, the method comprising:
controlling a motion of a robot based on an input of a user; and
measuring a magnitude of a reaction force of an object in contact with the robot based on the motion, through a sensor attached to the robot.

10. The method of claim 9, further comprising:
determining a magnitude and a direction of a feedback to be provided to the user, based on the magnitude and a direction of the reaction force of the object.

11. The method of claim 9, further comprising:
determining a property value of the object, based on the magnitude and a direction of the reaction force of the object;
determining a state of the object, based on reference information indicating state information of the object based on property values of the object, and the determined property value of the object; and
displaying, to the user, the state of the object, or a name of a disease related to the state of the object.

12. The method of claim 11, wherein the displaying comprises displaying the magnitude and the direction of the reaction force of the object, in a visual manner.

13. The method of claim 9, wherein the measuring comprises measuring a magnitude of a force fed back to a gripper of the robot by an object being gripped by the gripper, through a sensor attached to an internal side of the gripper gripping the object.

14. The method of claim 9, wherein the measuring comprises measuring a magnitude of a force fed back to an external side of a gripper of the robot by an object in contact with the external side of the gripper, through a sensor attached to the external side of the gripper.

15. The method of claim 9, wherein the measuring comprises measuring a magnitude of a force fed back to a probe of the robot by the object, or a shape of a surface of the object, through a sensor attached to the probe.

16. The method of claim 9, wherein the sensor measures the magnitude of the reaction force of the object, with respect to each of an X-axial direction, a Y-axial direction, and a Z-axial direction.

17. A non-transitory computer-readable medium comprising a program for instructing a computer to perform the method of claim 9.

18. The method of claim 11, wherein determining the property value comprises comparing a force applied to the object by the robot to the reaction force of the object.

19. The method of claim 11, wherein the property value comprises elasticity.

20. The method of claim 11, wherein the state information indicates a state of the object when the property value of the object is within a predetermined range.
